(19) United States
(12) Patent Application Publication
(10) Pub. No.: US 2003/0124083 A1
(43) Pub. Date: Jul. 3, 2003

(54) Non-soap based shaving and moisturizing composition

(75) Inventors: Steve Filipski, Addison, TX (US); William Basinski, Plano, TX (US)

Correspondence Address: JEFFER, MANGELS, BUTLER & MARMARO, LLP, 1900 AVENUE OF THE STARS, 7TH FLOOR, LOS ANGELES, CA, 90067, US

(73) Assignee: Woodridge Labs, Inc.

(21) Appl. No.: 10/038,830

(22) Filed: Dec. 28, 2001

(51) Int. Cl. A61K 31/728; A61K 7/15

(52) U.S. Cl. 424/73; 514/54

(57) ABSTRACT

A soap-free shaving and moisturizing composition which maintains superior performance attributes while avoiding the harshness and drying associated with soap-based shave preparations is described. The shaving and moisturizing composition preferably comprises deionized water, a humectant, a water-based silicone lubricant, and sodium hyaluronate. The composition can also contain a conditioner, preservatives, allantoin, and various heavy alkaloid extracts. The present invention is a single shaving composition that performs as a shave cream, an after shave, and a moisturizer. Applying the composition does not require the application of water on the shave area or that water be added to the shaving composition, however water can be applied if desired. In addition, use of the shaving composition does not require a hydration wait time between applying the composition and shaving. Once shaving is complete, any residual shaving composition on the shaved area is rubbed into the skin and acts as an after shave and a long-term moisturizer. Shaving can be performed by using a multiple blade razor having an open flow design, thereby reducing resistance while shaving and decreasing dryness and irritation of the skin.

### FIELD OF THE INVENTION

The present invention relates generally to a method and composition for improving the shaving process. More particularly, the invention relates to a composition which allows for shaving with minimal use of water and minimal wait time for the softening of hair before shaving whereby the entire shaving process is made more efficient.

### BACKGROUND OF THE INVENTION

Experience and testing have shown that the shaving process is not as efficient as it should be resulting in uncomfortable shaves, use of excess water, and general inconvenience in the shaving process. For example, for a comfortable shave, it is important that the hair fiber be softened as much as possible before the hair is cut. To achieve this, shaving is often performed while showering, adding up to six to ten minutes of additional time in which hot water is consumed, solely for the purpose of shaving.

The majority of persons who shave do so outside of the shower where applications of water and shaving preparations normally requires three to four minutes to fully hydrate and soften the hair, thereby giving the least resistance to cutting and giving the most comfortable shave. However, most shavers are not in the habit of waiting that long for hydration and typically begin shaving within a minute or less of applying the shaving preparation. At the completion of the cutting stage of shaving, shavers must apply water to the shaved area to rinse off remaining shave preparation. This often results in water falling outside the sink, generally making the shaving process inconvenient.

Finally, using soap-based shaving compositions can leave the skin dry. Even if a humectant is used to prevent drying, repeated use over extended periods can lead to overdrying. Shavers, particularly men, are often not in the habit of applying moisturizing agents to the shaved area to cure the drying. While some type of aftershave is commonly used, an application of some type of moisturizer is often not taken for various reasons. One is that it simply adds more time to a process already viewed as too time consuming. Another is that such application of a “moisturizing cream” has not generally been widely promoted or marketed, and accepted, among male shavers.

### SUMMARY OF THE PREFERRED EMBODIMENTS

The present invention comprises a soap-free shaving and moisturizing composition which maintains superior performance attributes while avoiding the harshness and drying associated with soap-based shave preparations. In one preferred embodiment of the present invention, the shaving and moisturizing composition comprises water, a water-based silicone lubricant, and sodium hyaluronate. In a much preferred embodiment, the composition includes a humectant, a conditioner, and allantoin in addition to water, a silicone lubricant and sodium hyaluronate. In another much preferred embodiment, the composition also includes preservatives and various heavy alkaloid extracts.

In another aspect of the present invention, a method of shaving is described. A shaving and moisturizing composition is applied to a dry or wet skin surface area. Hair on the surface area is cut using a razor blade immediately after the composition is applied to the surface area. Once shaving is complete, residual shaving composition on the surface area is manually rubbed into the surface area without the need for applying additional liquid to the surface area, although water or other liquids, such as after-shave, can be applied. The compositions of the present thus perform as an after-shave and a long-term moisturizer.

In another aspect of the invention, a method of shaving using a multiple blade razor is described. After hydrating the shave area, such as after showering, the shave area is dried and the shaving composition of the present invention is applied. A multiple blade razor having an open flow design is used to shave immediately after the shaving composition is applied. The open flow design of the razor blade and the properties of the shaving composition requires that reduced amount of water is needed to rinse the razor thereby extending the useful life of the razor blade.

### DETAILED DESCRIPTION OF THE PREFERRED EMBODIMENTS

Reference will now be made in detail to preferred embodiments of the invention. While the invention will be described in conjunction with its preferred embodiments, it will be understood that it is not intended to limit the invention to one preferred embodiment. To the contrary, the invention is intended to cover alternatives, modifications, and equivalents as may be included within the spirit and scope of the invention as defined by the appended claims.

The present invention is a single shaving composition that performs as a shave cream, an after-shave, and a moisturizer. Applying the composition does not require the application of water, however water can be applied on the shave area if desired by the shaver. Water need not be added to the shaving composition itself, which is primarily water based. A significantly reduced amount of water is needed to rinse or flush the shave composition and hair from one's razor blades compared to the amount of water typically needed with conventional shave compositions. In addition, use of the shaving composition does not require a significant hydration wait time between applying the composition and shaving. Shaving can begin seconds after the composition is applied to the shave area. Once shaving is complete, any residual composition on the shaved area can be rubbed into the skin thereby acting as an after-shave and a long-term moisturizer.

Components of a preferred embodiment of the present invention include deionized water, a humectant, to improve soothing of the skin, a water-based silicon lubricant and sodium hyaluronate, a derivative of hyaluronic acid. It should be appreciated that other salts of hyaluronic acid can also be used provided they are compatible with the other ingredients of the composition. In one preferred embodiment, the shaving composition contains, in percent by weight, (a) about 70 to 95% deionized water, about 2 to 10% of a humectant, such as butylene glycol, glycerin, sorbitol, propylene glycol, glycereth-7, glycereth-12, glycereth-26, glycereth-31, methyl gluceth-10, methyl gluceth-20, PEG-4, polyamino sugar condensate, polyquaternium-7, polyquaternium-22, polyquaternium-39, polyquaternium-47, PCA, sodium PCA, and UREA, or mixtures thereof, (b) about 0.25 to 5% of a water-based silicone lubricant, such as PEG8 dimethicone, dimenthicone copolyol, cyclomethicone, BIS-PEG/PPG-20/20 dimethicone, PEG/PPG-20/6 dimethicone, PEG-14 dimethicone, glyceryl polymethacrylate, hydrogenated vegetable oil, jojoba esters, mineral oil, octyl palmitate, petrolatum, and shea butter or mixtures thereof, or about 0.5 to 10% of a non-water based lubricant such as polytetrafluoroethylene (PTFE), also known as Teflonâ, for example, the micro BB version of PTFE or a liquid version having a lower molecular weight, (c) about 0.01 to 5% sodium hyalurononate or equivilent salts of hyaluronic acid, (d) about 0.05 to 0.5% of a conditioner, for making the hair shaft softer, such as panthenol, behentrimonium chloride, ceremide 2, cetrimonium chloride, hydrolyzed animal protein, hydrolyzed collagen, hydrolyzed keratin, hydrolyzed soy protein, hydrolyzed wheat protein, keratin amino acids, laurtrimonium chloride, niacin, niacinimide, phytantriol, polyquaternium-4, polyquaternium-6, polyquaternium-7, polyquaternium-10, polyquaternium-11, and polyquaternium-22, or mixtures thereof, (e) about 0.1 to 1% preservatives to reduce bacteria in the shaved area, such as chlorphenesin, phenonip, benzalkonium chloride, benzethonium chloride, benzoic acid, benzyl alcohol, benzylparaben, butylparaben, chloroxylenol, diazolidinyl urea, dimethyl oxazolidine, and disodium EDTA, or mixtures thereof, and (f) about 0.05 to 0.5% allantoin. The foregoing composition functions as a shave cream, after shave and moisturizer. The composition is substantially free of soap and may be used without applying water to the shave area or to the composition (with the exception of rinsing the razor blade during the shave).

More preferably the composition will comprise about 75 to 90% deionized water, about 3 to 7% butylene glycol as a humectant, about 1 to 3% PEG8 dimethicone as a water-based silicone lubricant or about 2 to 7% PTFE as a non-water based lubricant, about 0.01 to 0.05% sodium hyaluronate, about 0.05 to 0.10% panthenol, about 0.05 to 0.10% chlorphensin, about 0.1 to 0.4% phenonip, about 3 to 5% Canadian Willow Herb Extract, about 0.001 to 0.01% licorice extract, and about 0.0001 to 0.001% olive leaf extract. These alkaloid-heavy extracts collectively perform anti-bleeding and anti-acne functions and more generally function as soothers.

A most preferred shaving composition will have about 70 to 86% deionized water, about 5 to 8% butylene glycol, about 2 to 4% PEG8 dimethicone or about 3 to 6% PTFE, about 0.10 to 1.0% sodium hyaluronate, about 0.2 to 0.5% panthenol, about 0.10 to 0.20% chlorphensin, about 0.5 to 1.0% phenonip, about 1 to 5% Canadian Willow Herb Extract, about 0.1 to 1.0% licorice extract, and about 0.05 to 0.10% olive leaf extract.

A key component to a comfortable shave is hydration of the skin and hair shaft. This can be achieved through the hydroscopic properties of sodium hyaluronate acid that holds water on the skin and draws moisture from air in contact with the surface of the skin. Within one to two minutes and preferably one to 60 seconds after application, the skin is typically hydrated sufficently to comfortably shave. Sodium hyaluronate allows the skin to be replenished with moisture at a cellular level. It is a copolymer which provides lubricity and performs as a long-term moisturizer. The hydroscopic properties of sodium hyaluronate allow the retention of water and attracts water from the atmosphere to replenish the skin. Polymers of acetylglycosamine and glucuronic acid have similar properties and can be used to achieve hydration of the skin and perform as long-term moisturizers. Sodium hyaluronate, typically made from bacteria and yeast cultures, is available from Genzyme of Massachusetts.

Given that water need not be applied to the skin, traditional conditioners that need to be rinsed off, such as steric acid or palometic acid, are not used.

The following examples illustrate representative shaving and moisturizing compositions of the present invention. All parts and percentages are by weight.

Examples

| Ingredient (wt%) | 1(wt %) | 2(wt %) | 3(wt %) | 4(wt %) |
|---|---|---|---|---|
| Dionized Water | 87.379 | 80 | 95.0979 | 97.8897 |
| Butylene Glycol | 5.0 | 8.0 | 3.0 | 1.0 |
| PEG8 Dimethcone | 2.0 | .40 | 1.0 | 5 |
| Sodium Hyaluronate | .10 | 1.0 | .05 | .01 |
| Panthenol | .20 | 5 | 1 | .05 |
| Chlorphensin | .20 | 2 | .15 | 1 |
| Phenonip | .60 | .60 | 5 | 4 |
| Allantoin | .20 | .5 | 1 | .05 |
| Canadian Willow | .01 | 5.0 | .001 | .0001 |
| Herb Extract | | | | |
| Licorice Extract | .01 | 1 | .0001 | .0001 |
| Olive Leaf Extract | .001 | 1 | .0001 | .0001 |

Other useful additives which may be utilized in the composition include skin freshening and soothing agents such as menthol, aloe, and collagen. The shaving and moisturizing composition of the present invention may be packaged in any dispenser suitable for dispensing post-foaming shave gels. These include aerosol containers with a barrier, such as a collapsible bag or piston, to separate the gel from the propellant required for expulsion, collapsible tubes, and pump or squeeze containers. Another useful additive is sunblock.

The shaving composition of the present invention can be used to shave in various ways. As mentioned above, shaving products are more effective when the hair has been softened or soaked by a shaving composition for typically a minimum of three minutes. However, individuals, particularly men, often do not wait this long before shaving, thus do not begin cutting the hair at the optimal time. In a preferred embodiment, the shaving composition of the present invention is applied on dry skin immediately or soon after showering or hydrating the shave area, such as being in a sauna, steam room or after swimming. In the case of showering, hydration of a shave area, such a man's face, lasts several minutes after completion of the shower. Thus, once the shaving composition is applied, the person shaving does not have to wait for a hydration period (e.g., three minutes) but can begin shaving immediately and yet have an optimal shave.

Moreover, the shaver does not have to re-apply water to the shave area before using the shaving composition. By not having to apply water to the shave area for re-wetting the face before shaving and washing the shave area with water after shaving is complete, much of the inconvenience of spilling water outside the sink or onto other parts of the body is eliminated. Additionally, given the cream-like texture of the shave composition rather than the conventional foam or gel-to-foam texture of most shaving compositions, the inconvenience of having to applying, rinse and wash off excess foam is eliminated. As described above, any residual shaving composition on the skin after shaving is rubbed into the skin thereby reducing dryness from soap or foams.

By applying the shaving composition containing sodium hyaluronate and a silicone lubricant, as described in detail above, onto the shave area, resistance to cutting the hair is reduced. By reducing the resistance, the “drag” encountered by shavers is similarly reduced which, in turn, results in fewer strokes of the razor blade and less irritation on the skin. In a preferred embodiment, the shaving composition is used in combination with a multiple blade razor having an “open flow” blade design. One example of such a razor is the Mach3 razor with three razor blades and the open flow blade design available from Gillette. The design of the Mach3 and other multiple blade razors allows for easy rinsing of the shaving composition from between the blades. Thus, the properties of the shaving composition of the present invention makes optimal use of new razor blade systems and designs allowing for a close shave with minimal drag or resistance. Moreover, razor blades can be used for a longer time before becoming dull or ineffective.

Although the foregoing invention has been described in some detail for purposes of clarity of understanding, it will be apparent that certain changes and modifications may be practiced within the scope of the appended claims. Accordingly, the present embodiments are to be considered as illustrative and not restrictive, and the invention is not to be limited to the details given herein, but may be modified within the scope and equivalents of the appended claims.

1. A shaving and moisturizing composition comprising about 70 to 95% water, about 2 to 10% humectant, about 0.25 to 5% silicone lubricant, and 0.01 to 5% sodium hyaluronate.

2. A shaving and moisturizing composition according to claim 1 comprising about 75 to 90% water, about 3 to 7% humectant, about 1 to 3% silicone lubricant, and 0.01 to 0.05% sodium hyaluronate.

3. A shaving and moisturizing composition according to claim 1 comprising about 70 to 87% water, about 5 to 8% humectant, about 2 to 4% silicone lubricant, and 0.1 to 1.0% sodium hyaluronate.

4. A shaving and moisturizing composition comprising about 70 to 95% water, about 2 to 10% butylene glycol, about 0.25 to 5% PEG8 dimethicone, 0.01 to 5% sodium hyaluronate, about 0.05 to 0.5% conditioner, about 0.1 to 1.0% preservative, and about 0.05 to 0.5% allantoin.

5. A shaving and moisturizing composition according to claim 4 comprising about 75 to 90% water, about 0.05 to 0.5% butylene glycol, about 1 to 3% PEG8 dimethicone, 0.05 to 2% sodium hyaluronate, about 0.08 to 0.4% panthenol, about 0.05 to 0.1% chlorphensesin, and about 0.05 to 0.5% allantoin.

6. A shaving and moisturizing composition comprising about 70 to 86% water, about 5 to 8% butylene glycol, about 2 to 4% PEG8 dimethicone, 0.1 to 1.0% sodium hyaluronate, about 0.2 to 0.5% panthenol, about 0.1 to 0.2% chlorphensesin, and about 0.5 to 1.0% phenonip, and about 1 to 5% Canadian Willow Herb extract.

7. A shaving and moisturizing composition comprising about 70 to 95% water, about 2 to 10% proplylene glycol, about 0.25 to 5% PEG8 dimethicone, 0.01 to 5% sodium hyaluronate, about 0.05 to 0.5% panthenol, about 0.1 to 1% chlorphensesin, about 3 to 5% Canadian Willow Herb extract, about 0.001 to 0.01% licorice extract, and about 0.0001 to 0.001% olive leaf extract.

8. A shaving and moisturizing composition according to claim 7 comprising about 75 to 90% water, about 3 to 7% proplylene glycol, about 1 to 3% PEG8 dimethicone, 0.01 to 0.05% sodium hyaluronate, about 0.05 to 0.1% panthenol, about 0.05 to 0.1% chlorphensesin, about 1 to 5% Canadian Willow Herb extract, about 0.1 to 1% licorice extract, and about 0.05 to 0.1% olive leaf extract.

9. A shaving and moisturizing composition comprising about 70 to 95% water, about 2 to 10% humectant, about 0.5 to 10% polytetrafluoroethylene, and 0.01 to 5% sodium hyaluronate.

10. A shaving and moisturizing composition according to claim 9 comprising about 75 to 90% water, about 3 to 7% humectant, about 2 to 7% polytetrafluoroethylene, and 0.01 to 0.05% sodium hyaluronate.

11. A shaving and moisturizing composition comprising about 70 to 87% water, about 5 to 8% humectant, about 4 to 6% polytetrafluoroethylene, and 0.1 to 1.0% sodium hyaluronate.

12. A method of shaving hair on a surface area comprising:
applying a shave composition to the surface area;
shaving hair on the surface area after applying the shave composition wherein a hydration time for the hair to soften is between one and two minutes; and
spreading residual shave composition on the surface area.

13. A method as recited in claim 9 further comprising applying water to the surface area.

14. A method as recited in claim 9 wherein the composition contains one or more alkaloid-heavy extracts.

15. A method as recited in claim 9 wherein the composition contains sunblock.

16. A method as recited in claim 9 wherein the hydration time for the hair to soften is between one second and 60 seconds.

17. A method of shaving hair on a surface area comprising:
applying a shave composition to the surface area;
shaving hair on the surface area immediately after applying the shave composition; and
spreading on the surface area residual shave composition.

18. A method of shaving hair from skin comprising:
applying a hyaluronic acid based composition to the skin.

19. A method of shaving, conditioning and moisturizing the skin, comprising applying a hyaluronic acid based composition to the skin, shaving before soaking the skin with water, and rubbing the residual composition into the skin.

20. A method of shaving comprising:
showering thereby hydrating hair on a surface area making the hair softer;
drying the hair on the surface area upon completion of showering;
applying a shaving composition to the surface area upon drying the hair within a time in which the hair on the surface area remains hydrated and without reapplying water to the surface area; and
cutting the hair on the surface area, such that cutting the hair is performed with reduced resistance.

21. A method as recited in claim 20 further comprising:
rubbing residual shaving composition on the surface area upon completion of cutting the hair.

22. A method of increasing the efficiency of a multiple blade razor, the method comprising:
applying a shaving composition containing sodium hyaluronate and a silicone lubricant onto a shaving area; and
shaving the shaving area using a multiple blade razor whereby resistance to cutting the hair on the shaving area is reduced and whereby a minimal amount of water is needed to rinse the multiple blade razor between shaving strokes.

23. A method as recited in claim 22 wherein applying a shaving composition further comprises hydrating the shaving area and clearing the shaving area of water.

24. A method as recited in claim 22 wherein shaving the shaving area using a multiple blade razor further comprises allowing the shaving composition to flow through an open flow blade design of the multiple blade razor.

25. A shaving kit comprising a multiple blade razor and a shaving composition containing sodium hyaluronate, a humectant, and a silicone lubricant.

26. A shaving kit as recited in claim 25 wherein the multiple blade razor is a Mach3 razor blade.

27. A shaving kit comprising a multiple blade razor and a shaving composition containing sodium hyaluronate, a humectant, polytetrafluoroethylene, and water.

28. A method of shaving using a Mach3 razor blade, the method comprising applying a shaving composition containing sodium hyaluronate, a humectant, and a silicone lubricant and using a reduced amount of water to rinse the Mach3 razor blade thereby increasing the number of times the razor blade can be used effectively.
